(19) United States
(12) Patent Application Publication
(10) Pub. No.: US 2013/0345475 A1
(43) Pub. Date: Dec. 26, 2013

(54) PROCESS FOR THE RACEMIZATION OF OPTICALLY ACTIVE ARYLALKYLAMINES

(71) Applicant: BASF SE, Ludwigshafen (DE)

(72) Inventor: Marcus Georg SCHREMS, Ludwigshafen (DE)

(21) Appl. No.: 13/922,619

(22) Filed: Jun. 20, 2013

Related U.S. Application Data
(60) Provisional application No. 61/663,647, filed on Jun. 25, 2012.

(30) Foreign Application Priority Data
Jun. 25, 2012 (EP) 12173439.6

(51) Int. Cl. C07C 209/68

(52) U.S. Cl. CPC C07C 209/68
USPC 564/302

(57) ABSTRACT

Many optically active amines are valuable pharmaceuticals and intermediates for the preparation of active compounds. It is frequently the case that only one of the two enantiomers is active or not harmful, so that isolation of this enantiomer from the racemic mixture is necessary. Processes for racemate resolution make it possible to separate racemic mixtures into their enantiomers. Here, it is useful to once again racemize the enantiomer which is not required and recirculate it to racemate resolution and thus improve the yield of the desired enantiomer. The present invention relates to processes for the racemization of optically active amines, in particular arylalkylamines, in the presence of hydrogen and a hydrogenation/dehydrogenation catalyst comprising nickel, cobalt and copper as active components at elevated temperature.

The present invention relates to a process for the racemization of optically active amines of the formula I

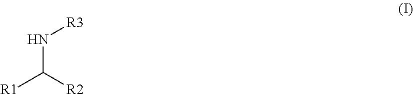

in the presence of hydrogen and a hydrogenation/dehydrogenation catalyst comprising nickel, cobalt and copper as active components at elevated temperature and at a hydrogen partial pressure of not more than 10 bar, where R1 and R2 are different, R1 is an aryl, arylalkyl or heteroaryl radical, R2 is a linear, branched or cyclic alkyl radical or a heterocyclic radical and R3 is a hydrogen atom, a linear, branched or cyclic alkyl radical, an arylalkyl radical, an aryl radical, a heteroaryl radical or a heterocyclic radical, where the radicals R1 and R2 may be joined to one another to form a ring structure and the radicals R1 to R3 may bear substituents selected from the group consisting of alkyl (linear, branched or cyclic), alkoxy, hydroxyl, halogen, aryloxy, amino, alkylamino and dialkylamino.

Optically active amines of the formula I are, for example, valuable pharmaceuticals and intermediates for the preparation of active compounds. It is frequently the case that only one of the two enantiomers (based on the asymmetric carbon atom shown in the formula I) is active or more active than the other enantiomers. Accordingly, the two enantiomers will here also be referred to as “desired enantiomer” and “undesired enantiomer”.

Optically active amines of the formula I can be obtained, for example, by resolution of racemates. One method for this is the classical racemate resolution as described, for example, by Faigl et al. (Tetrahedron: Asymmetry 2008, 19, 519). A further method is enzymatic racemate resolution as described, for example, in U.S. Pat. No. 5,728,876. Apart from the desired enantiomer, the undesired enantiomer is also obtained in both methods and the yield is therefore only 50% even in the ideal case. To increase the overall yield of the process and to reduce materials costs, it is therefore desirable to racemize the undesired enantiomer which is not required and subject it again to racemate resolution.

Such racemizations are described in the literature (Parvulescu et al., Top. Catal. 2010, 53, 931; Ebbers et al., Tetrahedron 1997, 53, 9417). Thus, for example, a method for the racemate resolution of 1-(1-naphthyl)ethylamine in which (R)-1-(1-naphthyl)ethylamine is obtained by classical racemate resolution by means of (R)-mandelic acid and the fraction of the undesired (S)-enantiomer which is not required and can also comprise residues of the (R)-enantiomer is racemized in a mixture of dimethyl sulfoxide and potassium hydroxide at 150-160° C., with the enantiomeric excess decreasing from 85% of (S) to 12% of (S), has been described (Mathad et al., Synthetic Communications 2011, 41, 341). Similar racemization processes are described in DE 4038356 A and WO 97/21662, in which a catalytic amount of a base is used. However, such processes are disadvantageous because of the large amount of solvent and base required.

Processes which allow optically active amines of the formula I to be racemized by means of a catalyst, in the ideal case in the presence of little or no solvent, are more advantageous. Thus, for example, homogeneous ruthenium catalysts by means of which optically active alkylaryl-amines can be racemized in the presence of a solvent have been described (Paetzold & Backvall, J. Am. Chem. Soc. 2005, 127, 17620). Disadvantages of these homogeneous catalyst systems are that such, generally expensive, catalysts can be separated from the product only with difficulty, large amounts of specifically dried solvents are required and only low space-time yields are obtained. Heterogeneous catalysts are therefore better suited to industrial processes.

EP 735018 A, EP 778260 A and EP 791569 A describe racemization processes in which optically active amines are firstly reacted with carbonyl compounds to form the corresponding imine compounds which are subsequently treated with base and finally hydrolyzed.

DE 2442845 A describes a process for the racemization of optically active amines by means of catalysts which comprise alkali metals as active component, for example precipitated on a solid support such as aluminum oxide.

The use of Raney cobalt or Raney nickel suspension catalysts, generally in the presence of hydrogen, for the racemization of optically active amines has also been described (Parvulescu et al., Adv. Synth. Catal. 2008, 350, 113; U.S. Pat. No. 4,990,666; U.S. Pat. No. 3,954,870; DE 2851039 A; DE 2903589 A; CH 351975 A). Here, U.S. Pat. No. 4,990,666 teaches that high temperatures, e.g. above 160° C., reduce the racemate yield.

The disadvantage of many such suspension catalysts is that they are used as a suspension having a proportion of water of usually about 50% and only a batch reaction is therefore possible. In addition, water interferes in the racemization since it leads to by-products which are often difficult to separate off by distillation (e.g. ketones from imines and possibly further reaction of the ketone to form the alcohol). It is therefore advantageous to remove the water as completely as possible. This is possible by washing the catalyst a number of times with a solvent before use. However, this complicated treatment generally does not lead to complete removal of the water, so that secondary reactions can still occur, albeit to a reduced extent.

Fixed-bed catalysts in the form of extrudates, spheres or pellets or other suitable shaped bodies, which are optionally activated by means of hydrogen, are therefore better suited. Some of these fixed-bed catalysts do not require any activation. The advantage of such catalysts is that they are virtually water-free and allow both a batch reaction and a continuous reaction.

WO 00/47546 discloses a process for the racemization of optically active amines by reaction of the optically active amine in the presence of hydrogen and a hydrogenation/dehydrogenation catalyst at elevated temperature, in which the reaction is carried out in the liquid phase and the catalyst comprises the catalytically active constituents copper, silver, iron, cobalt, nickel, ruthenium, rhodium, palladium, osmium, iridium and/or platinum and a support material selected from the group consisting of aluminum oxide, zirconium dioxide, titanium dioxide, carbon and oxygen-comprising compounds of silicon. WO 00/29357 also relates to a corresponding process for racemization in the gas phase.

Various palladium-comprising hydrogenation catalysts have been comprehensively described in the literature for use in the racemization of optically active amines (Parvulescu et al., Chem. Eur. J. 2007, 13, 2034; Parvulescu et al., Applied Catalyst A: General 2009, 368, 9).

U.S. Pat. No. 4,096,186 describes a process for the racemization of optically active amino alcohols, in which the amino alcohol is brought into contact with ammonia and hydrogen in the presence of a hydrogenation catalyst which preferably comprises cobalt. In the reaction of optically active 2-amino-1-butanol, a degree of racemization of only 63% is achieved at a racemate yield of not more than 97.6%. At a degree of racemization of 99%, on the other hand, a racemate yield of only 75.1% is achieved.

U.S. Pat. No. 6,060,624 relates to a process for the racemization of particular optically active primary ss-alkoxyalkylamines by reaction over a nickel or cobalt catalyst in the presence of hydrogen and ammonia.

EP 1215197 describes a process for preparing racemic mixtures from optically active amines using hydrogenation catalysts comprising copper and zinc at elevated temperature in the presence of hydrogen.

When particular metal catalysts or particular reaction conditions are used for the racemization, hydrodeamination of the amines can occur as undesirable secondary reaction (CH 351975 A). Thus, for example, the racemization of 1-(1-naphthyl)ethylamine in the presence of particular catalysts and/or under particular reaction conditions can lead to formation of 1-ethylnaphthalene, thus reducing the yield of amine.

Furthermore, an undesirable ring hydrogenation of the aryl group is frequently observed as a secondary reaction in the racemization of aryl-comprising optically active amines when using metal catalysts, particularly when the aryl group is an aromatic having fused ring systems. An example of such a fused ring system is the naphthalene radical. Thus, the reaction of 1-(1-naphthyl)ethylamine with hydrogen in the presence of a metal catalyst often forms various 1-(1-tetrahydronaphthyl)ethylamines as by-products. These can be separated off from 1-(1-naphthyl)ethylamine by distillation only with difficulty and with a high loss of yield.

It was an object of the present invention to discover an improved economical process for the racemization of optically active amines, in which the process product is obtained with a high degree of racemization and at the same time in a high racemization yield (racemate yield) and high space-time yield.

The process should allow the racemization of optically active amines having aryl groups, in particular aryl groups which have fused ring systems, with substantial avoidance of ring hydrogenation and/or other secondary reactions.

We have accordingly found a process for the racemization of optically active amines of the formula I

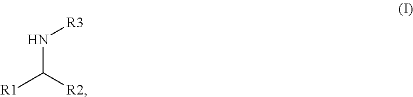

where R1 and R2 are different,
R1 is an aryl, arylalkyl or heteroaryl radical,
R2 is a linear, branched or cyclic alkyl radical or a heterocyclic radical and
R3 is a hydrogen atom, a linear, branched or cyclic alkyl radical, an arylalkyl radical, an aryl radical, a heteroaryl radical or a heterocyclic radical,
where the radicals R1 and R2 may be joined to one another to form a cyclic structure and the radicals may bear substituents selected from the group consisting of alkyl (linear, branched or cyclic), alkoxy, hydroxy, halogen, aryloxy, amino, alkylamino and dialkylamino,
   by reaction of the optically active amines of the formula I to form the racemization product in the presence of hydrogen and a hydrogenation/dehydrogenation catalyst at an elevated temperature of from 100 to 300° C., wherein the hydrogenation/dehydrogenation catalyst comprises the active components nickel, cobalt and copper which before activation by means of a reducing agent are preferably present entirely or partly in the form of their oxides and a hydrogen partial pressure of from 0.1 to 10 bar is maintained.

In a particular embodiment, the catalyst comprises at least one inert support material, preferably selected from the group consisting of aluminum oxide, silicon dioxide, titanium dioxide, zirconium oxide, magnesium oxide, iron oxide, cerium oxide and carbon. Support materials are, for example, Al₂O₃, SiO₂, ZrO₂, TiO₂, MgO, Fe₂O₃ or CeO₂. Carbon can, for example, be used in the form of activated carbon as support material. The catalyst particularly preferably comprises at least one inert oxidic support material selected from the group consisting of aluminum oxide, silicon dioxide and zirconium oxide, for example zirconium oxide.

The preparation of the support material is not subject to any particular restrictions.

In general, the oxidic support material can be prepared by, for example, precipitation from an aqueous solution comprising the corresponding nitrate (for example zirconium nitrate) by means of sodium carbonate and subsequent filtration, drying and optionally calcination of the precipitate obtained in this way.

As an alternative, a support-free catalyst (all-active catalyst) can also be used according to the invention.

The racemization according to the invention can be carried out in the gas phase or preferably in the liquid phase, batchwise or preferably continuously, and the catalyst is preferably arranged as a fixed bed in the reactor.

The racemization according to the invention can be carried out in the absence or preferably in the presence of the amine of the formula R3NH₂, where the radical R3 corresponds to the radical R3 of the optically active amine of the formula I (e.g. the amine ammonia in the case of racemization of optically active amines I in which R3=H).

If the racemization is carried out in the presence of the amine R3NH₂, use is generally made of up to 10 molar equivalents, preferably up to 5 molar equivalents, particularly preferably up to 3 molar equivalents, for example from 1 to 3 molar equivalents, of the amine R3NH₂ based on the amine of the formula I. The R3NH₂ excess can also be greater than 10 molar equivalents based on the amine of the formula I.

The reaction is carried out at a hydrogen partial pressure of from 0.1 to 10 bar, preferably from 0.1 to 7 bar, more preferably from 1 to 7 bar. It can be carried out in the presence of an inert diluent which is gaseous under the reaction conditions selected, e.g. nitrogen and/or argon.

When the process of the invention is carried out in the gas phase, the optically active amine of the formula I is continuously passed in gaseous form in a gas stream which comprises hydrogen and advantageously the amine R3NH₂ and is sufficiently large to effect vaporization over the catalyst in a reactor, for example an externally heated tube reactor.

Here, the flow into the fixed bed of catalyst can be either from above or from below. The gas stream required is preferably obtained by means of a gas recycle mode of operation, with, for example, an amount of recycled gas of from about 5 to 10 m³/h (volume converted to stp) and an amount of offgas of from about 250 to 350 l/h being set at a catalyst bed volume of 1 l. The space velocity over the catalyst is generally in the range from 0.1 to 2, preferably from 0.1 to 1, particularly preferably from 0.3 to 0.8, kg of amine of the formula I per liter of catalyst (bed volume) and hour.

When the process of the invention is carried out in the liquid phase, the optically active amine of the formula I is passed in liquid form in the presence of hydrogen and advantageously the amine R3NH₂ over the catalyst which is usually located in a preferably externally heated fixed-bed reactor, e.g. a tube reactor.

When the racemization is carried out in a tube reactor, the flow into the fixed catalyst bed can be from above (e.g. downflow mode) or from below (upflow mode). A gas recycle mode of operation is advantageous, with, for example, an amount of recycle gas of from about 0.01 to 1 m³/h (volume converted to stp) and an amount of offgas of from about 10 to 300 l/h being set at a catalyst bed volume of 1 l. The space velocity over the catalyst is generally in the range from 0.05 to 2, preferably from 0.1 to 1, particularly preferably from 0.2 to 0.6, kg of amine of the formula I per liter of catalyst (bed volume) and hour.

The temperatures selected for the racemization in the liquid phase and in the gas phase are in the range from 100 to 300° C., preferably from 150 to 250° C., particularly preferably from 160 to 220° C., very particularly preferably from 170 to 210° C., preferably until the ee reaches or goes below the desired value of for example 30%, preferably 10%, particularly preferably 5%. When the racemization is carried out batchwise, the racemization batch is usually subjected to the racemization conditions according to the invention for a period of from 2 to 24 hours.

The racemization of the optically active amine of the formula I in the liquid phase can be carried out in the presence of an inert diluent or solvent which is liquid under the reaction conditions selected, e.g. tetrahydrofuran, dioxane, N-methylpyrrolidone and/or ethylene glycol dimethyl ether.

Both when the process is carried out in the gas phase and when it is carried out in the liquid phase, the use of higher temperatures, higher total pressures and higher space velocities over the catalyst than those indicated above is possible.

After the reaction output has advantageously been depressurized, the hydrogen, any amine of the formula R3NH₂ used and any diluent or solvent used are removed therefrom (e.g. by distillation), with these being able to be recirculated, and the resulting cooled crude reaction product, which comprises essentially racemic amine of the formula I, is purified, for example by fractional rectification at atmospheric pressure or under reduced pressure.

For example, the racemization of 1-(1-naphthyl)ethylamine ((R or S)-NAPEA) (R1=1-naphthyl, R2=methyl, R3=H) can be carried out by the process of the invention.

The hydrogenation/dehydrogenation catalyst used according to the invention comprises, as active components, the elements nickel, cobalt and copper which before activation by means of a reducing agent are preferably present entirely or partly in the form of their oxides.

Suitable hydrogenation/dehydrogenation catalysts are described, for example, in EP 382049 A, EP 963975 A and EP 1106600B.

The hydrogenation/dehydrogenation catalysts can be used as supported catalyst in which the active components (nickel, cobalt and copper, or oxides thereof, optionally supplemented by one or more further active components) have been applied to a support material. The method of application is not subject to any restrictions.

For example, a nickel salt solution, a cobalt salt solution and a copper salt solution or a solution comprising nickel, cobalt and copper salts can be applied in one or more impregnation stages to the previously produced support in the form of powder, spheres, extrudates or pellets. The impregnated support is subsequently dried and optionally calcined. Such impregnation processes are, for example, described in EP 599180 A, EP 673918A.

It is also possible for a nickel salt solution, a cobalt salt solution and a copper salt solution or a solution comprising nickel, cobalt and copper salts to be applied by precipitation to the previously produced support which is, in a particularly preferred embodiment, present as powder in an aqueous suspension. The precipitation is carried out by the methods known in the prior art.

The hydrogenation/dehydrogenation catalysts can also be used as all-active catalyst in which the active components (nickel, cobalt and copper or oxides thereof, optionally supplemented by one or more further active components) are combined without support material.

Such support-free all-active catalysts can, for example, be prepared by precipitation of the active components from a nickel salt solution, a cobalt salt solution and a copper salt solution or a solution comprising nickel, cobalt and copper salts in the absence of a support, optionally in the presence of one or more further active components (for example likewise as solution or solution constituent).

The precipitates obtained from the precipitation process are generally filtered in a conventional way and preferably washed free of alkali, as described, for example, in DE 198 09 418 A, and dried at temperatures of from 50 to 150° C., preferably at about 120° C.

The nickel-, cobalt- and copper-comprising hydrogenation/dehydrogenation catalyst is preferably calcined before use in the racemization process of the invention. The calcination is carried out for, for example, from 0.5 to 10 hours at a temperature of generally from 200 to 600° C., in particular from 300 to 500° C.

As starting materials for production of the catalyst, it is in principle possible to use all copper(I) and/or copper(II) salts which are soluble in the solvents used for application, for example nitrates, carbonates, acetates, oxalates or ammonium complexes, and corresponding nickel and cobalt salts. Particular preference is given to using nickel nitrate, cobalt nitrate and copper nitrate.

For the process of the invention, the above-described dried and preferably calcined catalyst powder is preferably pressed to form pellets, rings, annular pellets, extrudates, honeycomb bodies or similar shaped bodies. All suitable methods known from the prior art can be used for this purpose.

The hydrogenation/dehydrogenation catalyst used according to the invention preferably comprises, after drying and before activation by means of a reducing agent, at least 5% by weight, preferably from 20 to 75% by weight, particularly preferably from 30 to 50% by weight, of oxygen-comprising compounds of nickel, calculated as NiO, at least 5% by weight, preferably from 20 to 75% by weight, particularly preferably from 30 to 50% by weight, of oxygen-comprising compounds of cobalt, calculated as CoO, at least 3% by weight, preferably from 5 to 50% by weight, particularly preferably from 10 to 30% by weight, of oxygen-comprising compounds of copper, calculated as CuO, in each case based on the sum of the oxygen-comprising compounds of nickel, cobalt and copper (in each case calculated as NiO, CoO and CuO, respectively).

The oxygen-comprising compounds of nickel, cobalt and copper (calculated as NiO, CoO and CuO, respectively) in toto preferably make up from 5 to 90% by weight, in particular from 15 to 80% by weight, of the total mass of the catalyst (including any further active components such as zirconium oxide or molybdenum oxide, any support materials, any additives and any shaping aids) after drying and before activation by means of a reducing agent.

In a particular embodiment, the hydrogenation/dehydrogenation catalyst used according to the invention additionally comprises at least one further active component selected from the group consisting of zirconium, molybdenum and palladium which, before activation by means of a reducing agent, is present entirely or partly in the form of oxides thereof; preference is given to using zirconium oxide as further active component. If molybdenum oxide and/or palladium oxide is/are used as further active component, these are preferably used in a proportion of in each case from 0.01 to 15% by weight, in particular from 0.1 to 5% by weight, in each case based on the sum of the oxygen-comprising compounds of nickel, cobalt and copper (in each case calculated as NiO, CoO and CuO, respectively) after drying and before activation by means of a reducing agent. If zirconium oxide is used as further active component, this is preferably used in a proportion of in each case from 1 to 75% by weight, in particular from 10 to 60% by weight, based on the sum of the oxygen-comprising compounds of nickel, cobalt and copper (in each case calculated as NiO, CoO and CuO, respectively) after drying and before activation by means of a reducing agent.

These additional active components can likewise be introduced into the catalyst material by means of the known precipitation or impregnation methods.

In the embodiment as supported catalyst, the hydrogenation/dehydrogenation catalyst used according to the invention preferably comprises, after drying and before activation by means of a reducing agent, in toto from 25 to 600% by weight, in particular from 200 to 400% by weight, of support materials selected from the group consisting of aluminum oxide, silicon dioxide, zirconium oxide, titanium dioxide, magnesium oxide, iron oxide and cerium oxide, in each case calculated as Al₂O₃, SiO₂, ZrO₂, TiO₂, MgO, Fe₂O₃ or CeO₂, based on the sum of the oxygen-comprising compounds of nickel, cobalt and copper (in each case calculated as NiO, CoO and CuO, respectively). In a further preferred embodiment, the hydrogenation/dehydrogenation catalyst used according to the invention comprises, after drying and before activation by means of a reducing agent, in toto from 25 to 600% by weight, in particular from 200 to 400% by weight, of support materials selected from the group consisting of aluminum oxide, silicon dioxide and zirconium oxide, in each case calculated as Al₂O₃, SiO₂, or ZrO₂, based on the sum of the oxygen-comprising compounds of nickel, cobalt and copper (in each case calculated as NiO, CoO and CuO, respectively). In a further preferred embodiment, the hydrogenation/-dehydrogenation catalyst used according to the invention comprises, after drying and before activation by means of a reducing agent, from 25 to 600% by weight, in particular from 200 to 400% by weight, of zirconium oxide as support material (calculated as ZrO₂), based on the sum of the oxygen-comprising compounds of nickel, cobalt and copper (in each case calculated as NiO, CoO and CuO, respectively).

In a particular embodiment, the hydrogenation/dehydrogenation catalyst used according to the invention does not comprise any zinc-comprising compound (including metallic zinc) or at least comprises less than 1% by weight, preferably less than 0.1% by weight, thereof (calculated as ZnO), based on the sum of the oxygen-comprising compounds of nickel, cobalt and copper (in each case calculated as NiO, CoO and CuO, respectively) after drying and before activation by means of a reducing agent.

In the production of shaped bodies composed of the catalysts used in the process of the invention, it is possible to add at least one additive selected from the group consisting of pulverulent copper, copper platelets and pulverulent cement to the catalyst material (active components and optionally support materials) before shaping in order to obtain better catalyst performance (higher activity, selectivity, mechanical and chemical stability).

As cement, preference is given to using a high-alumina cement. The high-alumina cement particularly preferably consists essentially of aluminum oxide and calcium oxide and particularly preferably comprises from about 75 to 85% by weight of aluminum oxide and from about 15 to 25% by weight of calcium oxide. It is also possible to use a cement based on magnesium oxide/aluminum oxide, calcium oxide/silicon oxide and calcium oxide/aluminum oxide/iron oxide.

For example, the at least one additive selected from the group consisting of pulverulent copper, copper platelets and pulverulent cement can be added in a proportion of in each case from 1 to 40% by weight, preferably from 2 to 30% by weight and particularly preferably from 5 to 20% by weight, in each case based on the sum of the oxygen-comprising compounds of nickel, cobalt and copper (in each case calculated as NiO, CoO and CuO, respectively) after drying and before activation by means of a reducing agent.

Preferred additives are pulverulent copper and/or copper platelets. The particularly preferred additive is copper platelets.

In the production of shaped bodies composed of the catalysts used in the process of the invention, it is possible to add one or more shaping aids to the catalyst material (active components and optionally support materials) before shaping. The addition of the shaping aids can be carried out before, after or simultaneously with the addition of the above-mentioned additives when both one or more of these additives and also one or more shaping aids are used. Customary shaping aids are graphite and stearic acid. The preferred shaping aid is graphite.

In general, one or more shaping aids selected from the group consisting of graphite and stearic acid are added in a proportion of in each case from 1 to 40% by weight, preferably from 2 to 30% by weight and particularly preferably from 2 to 20% by weight, in each case based on the sum of the oxygen-comprising compounds of nickel, cobalt and copper (in each case calculated as NiO, CoO and CuO, respectively) after drying and before activation by means of a reducing agent.

After addition of the additives and/or shaping aids to the catalyst material, the shaped catalyst body obtained after shaping can optionally be calcined at least once, in general for a period of from 0.5 to 10 hours, preferably from 0.5 to 2 hours. The temperature in this at least one calcination step is generally in the range from 200 to 600° C., in particular from 300 to 500° C.

The oxidic hydrogenation/dehydrogenation catalyst used according to the invention can be used in uncalcined form or preferably in calcined form.

The oxidic hydrogenation/dehydrogenation catalyst used according to the invention can be used after activation or without activation. Preference is given to activating the catalyst before use.

In a preferred embodiment, the shaped catalyst body is activated by treatment with reducing media, for example by heating in an atmosphere of reducing gases such as hydrogen or hydrogen/inert gas mixtures (e.g. hydrogen/nitrogen mixtures), preferably at temperatures in the range from 100 to 500° C., before use. Activation is either carried out beforehand in a reduction oven or after installation in the reactor. If the catalyst has been activated beforehand in a reduction oven, it is installed in the reactor and supplied directly under hydrogen pressure with the optically active amine of the formula I to be racemized.

When the shaped body is used as catalyst in the oxidic form, it is generally prereduced by means of reducing gases, for example hydrogen, preferably hydrogen/inert gas mixtures, in particular hydrogen/nitrogen mixtures, preferably at temperatures in the range from 100 to 500° C., particularly preferably in the range from 150 to 350° C. (activation), before being supplied with the optically active amine of the formula I to be racemized. Preference is given to using a mixture having a proportion of hydrogen in the range from 1 to 100% by volume, particularly preferably in the range from 1 to 50% by volume.

The radicals R1 and R2 of the amine of the formula I are different. R1 is an aryl, arylalkyl or heteroaryl radical, R2 is a linear, branched or cyclic alkyl radical or a heterocyclic radical and R3 is a hydrogen atom, a linear, branched or cyclic alkyl radical, an arylalkyl radical, an aryl radical, a heteroaryl radical or a heterocyclic radical, where the radicals may also bear substituents selected from the group consisting of alkyl (linear, branched or cyclic), alkoxy, hydroxy, halogen, aryloxy, amino, alkylamino and dialkylamino which are inert under the reaction conditions. The radicals R1 and R2 may be joined to one another to form a cyclic structure, for example in the case of 1-aminoindane and 1-aminotetralin.

### R1 is

an arylalkyl radical, preferably C7-20-arylalkyl, such as benzyl, 1-phenethyl, 2-phenethyl, 1-naphthylmethyl, 2-naphthylmethyl, phenanthrylmethyl, 4-tert-butyl-phenyl-methyl, 1-phenylpropyl, 2-phenylpropyl, 3-phenylpropyl, 1-phenylbutyl, 2-phenylbutyl, 3-phenylbutyl or 4-phenylbutyl,
   in particular such as 1-naphthylmethyl, 2-naphthylmethyl or phenanthrylmethyl,
an aryl radical, preferably C6-20-aryl, such as phenyl, 1-naphthyl, 2-naphthyl, 1-anthryl, 2-anthryl, 9-anthryl, in particular 1-naphthyl, 2-naphthyl, 1-anthryl, 2-anthryl or 9-anthryl, or
a heteroaryl radical, preferably C3-15-heteroaryl such as 2-pyridinyl, 3-pyridinyl, 4-pyridinyl, quinolinyl, pyrazinyl, pyrrol-3-yl, thienyl, imidazol-2-yl, 2-furanyl or 3-furanyl.
   R1 is preferably
an arylalkyl radical having 11-20 carbon atoms and at least one fused ring system, e.g. 1-naphthylmethyl, 2-naphthylmethyl, phenanthrylmethyl,
an aryl radical having 10-20 carbon atoms and at least one fused ring system, e.g. 1-naphthyl, 2-naphthyl, 1-anthryl, 2-anthryl, 9-anthryl, in particular 1-naphthyl or 2-naphthyl, or
a heteroaryl radical having 8-15 carbon atoms and at least one fused ring system, e.g. quinolinyl or isoquinolinyl.

### R2 is

a linear, branched or cyclic alkyl radical, preferably C1-20-alkyl,
   particularly preferably C1-12-alkyl such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, cyclohexylmethyl, n-octyl, 2-ethylhexyl, n-nonyl, isononyl, n-decyl, isodecyl, n-undecyl, n-dodecyl, isododecyl, cyclopropyl, cyclobutyl, cyclopentyl, cyclohexyl, cycloheptyl or cyclooctyl,
   very particularly preferably C1-8-alkyl such as methyl, ethyl, n-propyl, isopropyl, n-butyl, isobutyl, sec-butyl, tert-butyl, 2-ethylhexyl, cyclopentylmethyl, cyclohexylmethyl, cyclopentyl or cyclohexyl, or
a heterocyclic radical, preferably C3-15-heterocycloalkyl such as N-alkylpiperidin-3-yl, N-alkylpiperidin-4-yl, N,N′-dialkylpiperazin-2-yl, tetrahydrofuran-3-yl or N-alkyl-pyrrolidin-3-yl,

### R3 is

a hydrogen atom,
a linear, branched or cyclic alkyl radical, preferably C1-20-alkyl,
   particularly preferably C1-12-alkyl such as methyl, ethyl, n-propyl, isopropyl, n-butyl, isobutyl, sec-butyl, tert-butyl, n-pentyl, isopentyl, sec-pentyl, neopentyl, 1,2-dimethylpropyl, n-hexyl, isohexyl, sec-hexyl, cyclopentylmethyl, n-heptyl, isoheptyl, cyclohexylmethyl, n-octyl, 2-ethylhexyl, n-nonyl, isononyl, n-decyl, isodecyl, n-undecyl, n-dodecyl, isododecyl, cyclopropyl, cyclobutyl, cyclopentyl, cyclohexyl, cycloheptyl or cyclooctyl,
   very particularly preferably C1-8-alkyl such as methyl, ethyl, n-propyl, isopropyl, n-butyl, isobutyl, sec-butyl, tert-butyl, 2-ethylhexyl, cyclopentylmethyl, cyclohexylmethyl, cyclopentyl or cyclohexyl,
an arylalkyl radical, preferably C7-20-arylalkyl such as benzyl, 1-phenethyl, 2-phenethyl, 1-naphthylmethyl, 2-naphthylmethyl, phenanthrylmethyl, 4-tert-butylphenyl-methyl, 1-phenylpropyl, 2-phenylpropyl, 3-phenylpropyl, 1-phenylbutyl, 2-phenylbutyl, 3-phenylbutyl or 4-phenylbutyl,
   in particular 1-naphthylmethyl, 2-naphthylmethyl or phenanthrylmethyl,
an aryl radical, preferably C6-20-aryl such as phenyl, 1-naphthyl, 2-naphthyl, 1-anthryl, 2-anthryl, 9-anthryl, in particular 1-naphthyl, 2-naphthyl, 1-anthryl, 2-anthryl or 9-anthryl,
a heteroaryl radical, preferably C3-15-heteroaryl such as 2-pyridinyl, 3-pyridinyl, 4-pyridinyl, quinolinyl, pyrazinyl, pyrrol-3-yl, thienyl, imidazol-2-yl, 2-furanyl or 3-furanyl, or
a heterocyclic radical, preferably C3-15-heterocycloalkyl such as N-alkyl-piperidin-3-yl, N-alkylpiperidin-4-yl, N,N′-dialkylpiperazin-2-yl, tetrahydrofuran-3-yl or N-alkyl-pyrrolidin-3-yl.
   R3 is particularly preferably a hydrogen atom.

The radicals R1 to R3 can, independently of one another, bear substituents which are inert under the reaction conditions, preferably selected from the group consisting of C1-20-alkyl, C3-8-cycloalkyl, C1-20-alkoxy, hydroxy, halogen, C6-20-aryloxy, amino, C1-20-alkylamino and C2-20-dialkylamino, particularly preferably selected from the group consisting of C1-20-alkoxy, hydroxy, halogen, C6-20-aryloxy, amino, C1-20-alkylamino and C2-20-dialkylamino.

Here, the number of these substituents on the radicals R1 to R3 can be, depending on the type of radical, from 0 to 5, preferably from 0 to 3, in particular 0, 1 or 2. Possible substituents are, in particular:
C1-20-alkyl, as defined above,
C1-20-alkoxy, preferably C1-8-alkoxy such as methoxy, ethoxy, n-propoxy, isopropoxy, n-butoxy, isobutoxy, sec-butoxy, tert-butoxy, n-pentoxy, isopentoxy, sec-pentoxy, neopentoxy, 1,2-dimethylpropoxy, n-hexoxy, isohexoxy, sec-hexoxy, n-heptoxy, isoheptoxy, n-octoxy, isooctoxy,
   particularly preferably C1-4-alkoxy such as methoxy, ethoxy, n-propoxy, isopropoxy, n-butoxy, isobutoxy, sec-butoxy or tert-butoxy,
      hydroxy (—OH),
      halogen such as a chlorine atom or fluorine atom,
      C6-20-aryloxy such as phenoxy, 1-naphthoxy or 2-naphthoxy, preferably phenoxy, amino (—NH₂),
C1-20-alkylamino, preferably C1-12-alkylamino, in particular C1-8-alkylamino such as methylamino, ethylamino, n-propylamino, isopropylamino, n-butylamino, isobutylamino, tert-butylamino, cyclopentylamino or cyclohexylamino, or
C2-20-dialkylamino, preferably C2-12-dialkylamino, in particular C2-8-dialkylamino such as N,N-dimethylamino, N,N-diethylamino, N,N-di-n-propylamino, N,N-diisopropyl-amino, N,N-di-n-butylamino, N-ethyl-N-methylamino, N-methyl-N-propylamino or dicyclohexylamino.

For the purposes of the present invention, alkyl and alkoxy groups are saturated groups without C—C multiple bonds. Unless stated otherwise, for the purposes of the present invention, an alkyl group is a linear, branched or cyclic alkyl group.

Examples of amines of the formula I which can be used in the process of the invention are: 1-(1-naphthyl)ethylamine, 1-(2-naphthyl)ethylamine, 1-(1-naphthyl)propylamine, 1-(2-naphthyl)-propylamine, 1-(1-naphthyl)isobutylamine, 1-(2-naphthyl)isobutylamine, 1-(1-naphthyl)-n-butylamine, 1-(2-naphthyl)-n-butylamine, 1-phenylethylamine, 1-phenylpropylamine, 1-phenylisobutylamine, 1-phenyl-n-butylamine, 1-(3-methoxyphenyl)ethylamine, 1-(2-methoxyphenyl)-ethylamine, 1-(4-methoxyphenyl)ethylamine, 1-(3-methylphenyl)ethylamine, 1-(2-methylphenyl)-ethylamine, 1-(4-methylphenyl)ethylamine, 1-(3-chlorophenyl)ethylamine, 1-(2-chlorophenyl)-ethylamine, 1-(4-chlorophenyl)ethylamine, 1-(3-fluorophenyl)ethylamine, 1-(2-fluorophenyl)-ethylamine, 1-(4-fluorophenyl)ethylamine, 1-(3-hydroxyphenyl)ethylamine, 1-(2-hydroxyphenyl)-ethylamine, 1-(4-hydroxyphenyl)ethylamine, 1-(3-t-butylphenyl)ethylamine, 1-(2-t-butylphenyl)-ethylamine, 1-(4-t-butylphenyl)ethylamine, 2-amino-1-phenylpropane, 2-amino-1-(p-hydroxyphenyl)propane, 2-amino-1-(p-trifluoromethylphenyl)propane, 1-aminoindane, 1-aminotetralin, 2-aminotetralin.

Preferred amines of the formula I are those in which

R1 is an aryl radical, preferably a phenyl or naphthyl radical,
R2 is an alkyl radical, preferably a linear, branched or cyclic C1-12-alkyl radical, and
R3 is a hydrogen atom or an alkyl radical, preferably a hydrogen atom or a linear, branched or cyclic C1-12-alkyl radical, particularly preferably a hydrogen atom,
where the radicals R1 to R3 each bear, independently of one another, from 0 to 3 substituents selected from the group consisting of a linear C1-12-alkyl radical, a branched C1-12-alkyl radical, a cyclic C1-12-alkyl radical, a linear C1-12-alkoxy radical, a branched C1-12-alkoxy radical, a cyclic C1-12-alkoxy radical, hydroxy, fluoro and chloro and
the radicals R1 and R2 may be joined to one another to form a cyclic structure, for example in the case of 1-aminoindane and 1-aminotetralin. In a preferred embodiment, R1 is a phenyl radical. In another preferred embodiment, R1 is a naphthyl radical.

Particularly preferred amines of the formula I are 1-naphthylalkylamines in which R1=1-naphthyl or 2-naphthyl, R2=alkyl, in particular C1-20-alkyl, and R3=H.

Examples are 1-(1-naphthyl)ethylamine (1-NAPEA) and 1-(2-naphthyl)ethylamine.

In a particular variant, the undesired enantiomer of the amine of the formula I which is obtained by racemate resolution of the racemic amine of the formula I into the desired enantiomer and the undesired enantiomer of the amine of the formula I (based on the asymmetric carbon atom shown in formula I) is used as optically active amine of the formula I in the racemization process of the invention and the racemization product is returned to racemate resolution, so that the desired enantiomer of the amine of the formula I is obtained as end product of the overall process. In this context, the terms “undesired enantiomer” and “desired enantiomer” encompass fractions in which the respective enantiomer is present in a higher concentration compared to the other enantiomer.

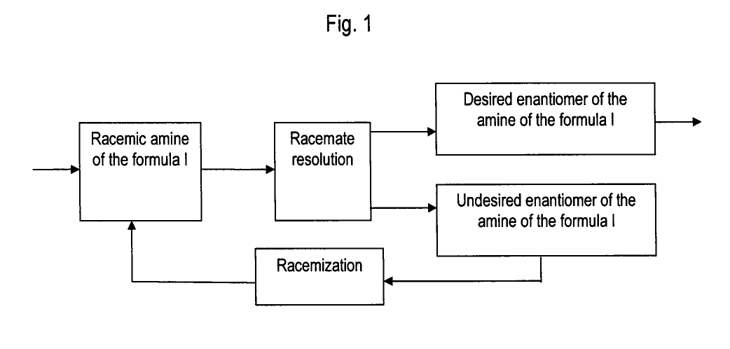

FIG. 1 shows a process of this type which combines racemate resolution and racemization in simplified form.

Racemate resolution is carried out by the methods known in the prior art. The racemic amine of the formula I can, for example, be converted by means of a chiral auxiliary acid (e.g. R-mandelic acid) into diastereomeric salts which can then be separated by means of fractional crystallization into the salts of the two enantiomers. A further possibility for the racemate resolution of racemic amines of the formula I is enantioselective acylation in the presence of a hydrolyase (WO 95/08636, WO 96/23894), where the acylating agent (e.g. ethyl methoxyacetate) and hydrolase (e.g. a lipase) are selected so that the amides derived from the enantiomers of the amine of the formula I are enzymatically cleaved with differing selectivity by the hydrolase. In a subsequent step, the amines can then be separated from the amides and the amides which have been separated off can be reconverted into the amine of the formula I (WO 97/10201). A further possibility for racemate resolution is chiral chromatography using a chiral separation medium on which the enantiomers to be separated have different retention times. In all methods of racemate resolution, a racemic mixture is separated into two fractions in which one of the two enantiomers is present in a higher concentration. To separate the enantiomers from one another as completely as possible, it is generally advantageous to repeat the separation step of racemate resolution.

The invention therefore also provides a process for isolating the desired enantiomer of the amine of the formula I (based on the asymmetric carbon atom shown in formula I) starting from the racemic amine of the formula I, which comprises the steps
(a) provision of the racemic amine of the formula I,
(b) separation of the racemic amine of the formula I by means of racemate resolution into a fraction A which comprises the major part of the desired enantiomer of the amine of the formula I and a fraction B which comprises the major part of the undesired enantiomer of the amine of the formula I,
(c) racemization of the amine of the formula I from the fraction B to form the racemic amine of the formula I in the presence of hydrogen and a hydrogenation/dehydrogenation catalyst at an elevated temperature of from 100 to 300° C. and
(d) recirculation of the racemic amine of the formula I obtained in step (c) to the racemate resolution of step (b),
   wherein the hydrogenation/dehydrogenation catalyst comprises the active components nickel, cobalt and copper which before activation by means of a reducing agent are preferably present entirely or partly in the form of their oxides
   and a hydrogen partial pressure of from 0.1 to 10 bar is maintained in the racemization. The radicals of the amine of the formula I are selected as described above. The racemization in step (c) is carried out as described above for the racemization according to the invention.

The ratio of desired enantiomer of the amine of the formula I to undesired enantiomer of the amine of the formula I in fraction A is preferably at least 60:40, particularly preferably at least 80:20 and very particularly preferably 90:10, corresponding to an enantiomeric excess (ee) of preferably at least 20%, particularly preferably at least 60% and very particularly preferably at least 80%.

The enantiomeric excess (ee) is given in percent by the following formula:

ee=[(m₊−m₋)/(m₊+m₋)]*100%,

where m₊ is the mole fraction of the excess enantiomer and m₋ is the mole fraction of the deficient enantiomer. Ideally, the product of a racemization has a low ee. The racemization according to the invention of optically active amines of the formula I preferably results in an ee of not more than 30%, particularly preferably not more than 10%, very particularly preferably not more than 5%.

Amines of the formula I which have one or more further asymmetric carbon atoms in addition to the asymmetric carbon atom shown in the formula I and which are thus diastereomers or diastereomeric mixtures can be reacted in the same way by means of the process of the invention. Accordingly, the terms “enantiomers”, “racemization” and “racemate resolution” are, for the purposes of the present invention, to be interpreted as encompassing diastereomers, isomerization or diastereomer separation when the process of the invention is applied to amines of the formula I which have one or more further asymmetric carbon atoms in addition to the asymmetric carbon atom shown in formula I. The racemization of the optically active amine of the formula I, or the desired and undesired enantiomers of the amines of the formula I and the racemic amine of the formula I are based on the asymmetric carbon atom shown in the formula I as a centre of clarity.

### EXAMPLES

### Example 1

### Preparation of an Ni/Co/Cu Oxide Catalyst

The catalyst was prepared as described for “catalyst A” in EP 963975 A. It had the composition: 28% by weight of Ni (calculated as NiO), 28% by weight of Co (calculated as CoO), 11% by weight of Cu (calculated as CuO) and 33% by weight of Zr (calculated as ZrO₂) based on the total mass of the catalyst (sum of the oxygen-comprising compounds of nickel, cobalt, copper and zirconium (in each case calculated as NiO, CoO, CuO or ZrO₂, respectively).

### Example 2

### Racemization of (R)-1-(1-naphthyl)ethylamine at 200° C.

An autoclave was charged with 4.5 g of the calcined catalyst from example 1 and closed. The autoclave was flushed twice with nitrogen and subsequently pressurized with hydrogen until a pressure of 50 bar had been reached. The autoclave was heated to 200° C. and on reaching the temperature hydrogen was injected in such an amount that a total pressure of 200 bar was reached in order to activate the catalyst. After two hours under these conditions, the autoclave was cooled and depressurized and (R)-1-(1-naphthyl)ethylamine (86.1 g, ee: 98.8%) was introduced into the closed autoclave by means of a pump. Ammonia (18.7 g) was subsequently injected as liquefied gas and the autoclave was heated to 200° C. 5 bar of hydrogen were injected in addition to the existing pressure of the autoclave (about 62 bar). After stirring for 12 hours, the autoclave was cooled to 40° C. and depressurized, and the reaction mixture was degassed and analyzed. According to gas-chromatographic analysis, the mixture comprised 5% of 1-ethylnaphthalene, 84% of 1-(1-naphthyl)ethylamine and 9% of a high boiler, presumably bis(1-naphthalen-1-ylethyl)amine. The proportion of remaining components was below 1%. The ee was 0%.

### Example 3

### Racemization of (R)-1-(1-naphthyl)ethylamine at 175° C.

The catalyst was activated as described in example 2, and the cooled and depressurized autoclave was then likewise charged with (R)-1-(1-naphthyl)ethylamine (86.1 g, ee: 98.8%) by means of a pump and ammonia (18.4 g) was subsequently injected as liquefied gas. The autoclave was heated to 175° C. 5 bar of hydrogen were injected in addition to the existing pressure of the autoclave (about 62 bar). After stirring for 12 hours, the autoclave was cooled to 40° C. and depressurized, and the reaction mixture was degassed and analyzed. According to gas-chromatographic analysis, the mixture comprised 1.5% of 1-ethylnaphthalene, 92% of 1-(1-naphthyl)ethylamine, <0.1% of ring-hydrogenated products and 4.3% of a high boiler, presumably bis(1-naphthalen-1-ylethyl)amine. The ee was 35% of (R).

### Example 4

### Racemization of (R)-1-(1-naphthyl)ethylamine at 175° C.

Example 4 was carried out and analyzed in the same way as example 3, except that only 17.2 g (0.1 mol; ee: 98.8%) of (R)-1-(1-naphthyl)ethylamine were used. According to gas-chromatographic analysis, the reaction mixture comprised 1.5% of 1-ethylnaphthalene, 87% of 1-(1-naphthyl)ethylamine, 0.3% of ring-hydrogenated products and 5.9% of a high boiler, presumably bis(1-naphthalen-1-ylethyl)amine. The ee was 31% of (R).

### Comparative Example 1

### Racemization of (R)-1-(1-naphthyl)ethylamine at 175° C. and a hydrogen partial pressure of 25 bar

Comparative example 1 was carried out and analyzed in the same way as example 3, except that 25 bar of hydrogen were injected. According to gas-chromatographic analysis, the reaction mixture comprised 4% of 1-ethylnaphthalene, 85% of 1-(1-naphthyl)ethylamine, 5.2% of ring-hydrogenated products and 0.5% of a high boiler, presumably bis(1-naphthalen-1-ylethyl)amine. The ee was 3% of (R).

### Comparative Example 2

### Racemization of (R)-1-(1-naphthyl)ethylamine at 200° C. over a Cu/Zn catalyst

The Cu/Zn catalyst was prepared as described in EP 1215197 A (example 1) and activated as described in example 2 before use. The cooled and depressurized autoclave was then likewise charged with (R)-1-(1-naphthyl)ethylamine (50 g, ee: >99%) by means of a pump and ammonia (10.5 g) was subsequently injected as liquefied gas. The autoclave was heated to 200° C. 5 bar of hydrogen were injected in addition to the existing pressure of the autoclave. After stirring for 12 hours, the autoclave was cooled to 40° C. and depressurized, and the reaction mixture was degassed and analyzed. The ee after the reaction was 99% of (R).

### Example 5

### Racemate resolution of 1-(1-naphthyl)ethylamine

The racemate resolution of 1-(1-naphthyl)ethylamine was carried out as described by K. Ditrich (Synthesis 2008, 14; 2283-2287). 1-(1-Naphthyl)ethylamine (17.2 g, 0.1 mol) and isopropyl methoxyacetate (19.8 g, 0.15 mol) were dissolved in diethyl ether (400 ml). After addition of immobilized lipase B from Candida antarctica (Novozyme 435®) (1 g), the turbid solution was stirred overnight at room temperature (30 rpm). The progress of the reaction was monitored by means of chiral HPLC. The reaction was stopped when an ee of 99.5% for the unreacted S-enantiomer and of 99.1% for the amide of the R-enantiomer was reached. Solid reaction residues were removed by filtration and washed with diethyl ether (50 ml). The combined filtrates were admixed with 5% strength aqueous HCl (50 ml) while stirring. After phase separation, the organic phase was washed with water (100 ml). The combined aqueous phases were reextracted with diethyl ether (3×70 ml) and the combined organic extracts were dried over sodium sulfate. After evaporation of the solvent and removal of unreacted isopropyl methoxyacetate by distillation, the amide of the R-enantiomer was obtained in the form of colorless crystals in a yield of 93%. An optically pure sample having a melting point of 80° C. could be obtained by recrystallization from cyclohexane. The combined aqueous extracts were made alkaline (pH 13) by addition of 50% strength aqueous NaOH while cooling in an ice bath. Diethyl ether (75 ml) was subsequently added and the phases were separated. The aqueous phase was extracted with diethyl ether (2×75 ml) and the combined extracts were dried over sodium sulfate. After evaporation of the solvent and purification by distillation, the S-enantiomer which remained was obtained in a yield of 92%.

1. A process for the racemization of an optically active amine of formula I 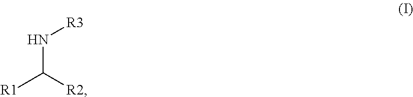
where R1 and R2 are different,
R1 is an aryl, arylalkyl or heteroaryl radical,
R2 is a linear, branched or cyclic alkyl radical or a heterocyclic radical and
R3 is a hydrogen atom, a linear, branched or cyclic alkyl radical, an arylalkyl radical, an aryl radical, a heteroaryl radical or a heterocyclic radical,
where the radicals R1 and R2 may be joined to one another to form a cyclic structure
and the radicals may comprise one or more substituents selected from the group consisting of alkyl, alkoxy, hydroxy, halogen, aryloxy, amino, alkylamino and dialkylamino,
the process comprising reacting the optically active amine of formula I, in the presence of hydrogen and a hydrogenation/dehydrogenation catalyst at an elevated temperature of from 100 to 300° C., to form a racemization product,
wherein the hydrogenation/dehydrogenation catalyst comprises as active components nickel, cobalt and copper, and a hydrogen partial pressure of from 0.1 to 10 bar is maintained during the reacting.

2. The process of claim 1, wherein the active components nickel, cobalt and copper of the hydrogenation/dehydrogenation catalyst are present entirely or partly in a form of oxides before being activated by a reducing agent.

3. The process of claim 1, wherein the hydrogenation/dehydrogenation catalyst comprises an inert support material.

4. The process of claim 1, wherein the hydrogenation/dehydrogenation catalyst additionally comprises at least one further active component selected from the group consisting of zirconium, molybdenum and palladium which further active component is present entirely or partly in a form of an oxide, before being activated by a reducing agent.

5. The process of claim 1, wherein the reacting is carried out in the presence of an amine of formula R3NH₂ in which R3 corresponds to the radical R3 of the optically active amine of formula I.

6. The process of claim 1, wherein the hydrogenation/dehydrogenation catalyst comprises less than 1% by weight of zinc-comprising compounds including metallic zinc, calculated as ZnO, based on a sum of oxygen-comprising compounds of nickel, cobalt and copper, in each case calculated as NiO, CoO and CuO, respectively, after drying and before being activated by a reducing agent.

7. The process of claim 1, wherein
R1 is a C7-20-arylalkyl radical, a C6-20-aryl radical or a C3-15-heteroaryl radical,
R2 is a linear, branched or cyclic C1-20-alkyl radical or a C3-15-heterocycloalkyl radical and
R3 is hydrogen, a C7-20-arylalkyl radical, a C6-20-aryl radical, a C3-15-heteroaryl radical, a linear, branched or cyclic C1-20-alkyl radical or a C3-15-heterocycloalkyl radical and
the radicals R1 to R3 comprise, independently of one another, from 0 to 5 substituents which are inert under the reaction conditions and are selected from the group consisting of C1-20-alkoxy, hydroxy, halogen, C6-20-aryloxy, amino, C1-20-alkylamino and C2-20-dialkylamino.

8. The process of claim 1, wherein
R1 is a C11-20-arylalkyl radical having at least one fused ring system, a C10-20-aryl radical having at least one fused ring system or a C8-15-heteroaryl radical having at least one fused ring system,
R2 is a linear, branched or cyclic C1-20-alkyl radical or a C3-15-heterocycloalkyl radical and
R3 is hydrogen, a C7-20-arylalkyl radical, a C6-20-aryl radical, a C3-15-heteroaryl radical, a linear, branched or cyclic C1-20-alkyl radical or a C3-15-heterocycloalkyl radical and
the radicals R1 to R3 comprise, independently of one another, from 0 to 5 substituents which are inert under the reaction conditions and are selected from the group consisting of C1-20-alkoxy, hydroxy, halogen, C6-20-aryloxy, amino, C1-20-alkylamino and C2-20-dialkylamino.

9. The process of claim 1, wherein the amine of formula I is selected from the group consisting of 1-(1-naphthyl)ethylamine, 1-(2-naphthyl)ethylamine, 1-(1-naphthyl)propylamine, 1-(2-naphthyl)propylamine, 1-(1-naphthyl)isobutylamine, 1-(2-naphthyl)isobutylamine, 1-(1-naphthyl)-n-butylamine, 1-(2-naphthyl)-n-butylamine, 1-phenylethylamine, 1-phenylpropylamine, 1-phenylisobutylamine, 1-phenyl-n-butylamine, 1-(3-methoxyphenyl)ethylamine, 1-(2-methoxyphenyl)ethylamine, 1-(4-methoxyphenyl)ethylamine, 1-(3-methylphenyl)ethylamine, 1-(2-methylphenyl)-ethylamine, 1-(4-methylphenyl)ethylamine, 1-(3-chlorophenyl)ethylamine, 1-(2-chlorophenyl)ethylamine, 1-(4-chlorophenyl)ethylamine, 1-(3-fluorophenyl)ethylamine, 1-(2-fluorophenyl)ethylamine, 1-(4-fluorophenyl)ethylamine, 1-(3-hydroxyphenyl)ethylamine, 1-(2-hydroxyphenyl)ethylamine, 1-(4-hydroxyphenyl)ethylamine, 1-(3-t-butylphenyl)ethylamine, 1-(2-t-butylphenyl)ethylamine, 1-(4-t-butylphenyl)ethylamine, 2-amino-1-phenylpropane, 2-amino-1-(p-hydroxyphenyl)propane, 2-amino-1-(p-trifluoromethylphenyl)propane, 1-aminoindane, 1-aminotetralin and 2-aminotetralin.

10. The process of claim 1, wherein the amine of formula I is selected from the group consisting of 1-(1-naphthyl)ethylamine, 1-(2-naphthyl)ethylamine, 1-(1-naphthyl)propylamine, 1-(2-naphthyl)propylamine, 1-(1-naphthyl)isobutylamine, 1-(2-naphthyl)isobutylamine, 1-(1-naphthyl)-n-butylamine and 1-(2-naphthyl)-n-butylamine.

11. The process of claim 1, which yields an undesired enantiomer of the amine of formula I and a desired enantiomer of the amine of formula I, wherein the process is repeated using the undesired enantiomer of the amine of formula I as the optically active amine of formula I, to obtain the desired enantiomer of the amine of formula I as an end product of the process.

12. A process for isolating a desired enantiomer of an amine of formula I, starting from a first racemic mixture of the amine 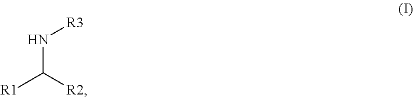
where R1 and R2 are different,
R1 is an aryl, arylalkyl or heteroaryl radical,
R2 is a linear, branched or cyclic alkyl radical or a heterocyclic radical and
R3 is a hydrogen atom, a linear, branched or cyclic alkyl radical, an arylalkyl radical, an aryl radical, a heteroaryl radical or a heterocyclic radical,
where the radicals R1 and R2 may be joined to one another to form a cyclic structure
and the radicals may comprise one or more substituents selected from the group consisting of linear alkyl (branched alkyl, cyclic alkyl, alkoxy, hydroxy, halogen, aryloxy, amino, alkylamino and dialkylamino,
the process comprising:
(a) separating the first racemic mixture of the amine by racemate resolution into a fraction A which comprises a major part of the desired enantiomer of the amine of formula I and a fraction B which comprises a major part of an undesired enantiomer of the amine of formula I,
(b) racemizing the amine of formula I from the fraction B to form a second racemic mixture of the amine in the presence of hydrogen and a hydrogenation/dehydrogenation catalyst at an elevated temperature of from 100 to 300° C., and
(c) recirculating the second racemic mixture of the amine obtained in (b) to (a),
wherein the hydrogenation/dehydrogenation catalyst comprises as active components nickel, cobalt and copper and a hydrogen partial pressure of from 0.1 to 10 bar is maintained during the racemizing.

13. The process of claim 12, wherein
R1 is a C7-20-arylalkyl radical, a C6-20-aryl radical or a C3-15-heteroaryl radical,
R2 is a linear, branched or cyclic C1-20-alkyl radical or a C3-15-heterocycloalkyl radical and
R3 is hydrogen, a C7-20-arylalkyl radical, a C6-20-aryl radical, a C3-15-heteroaryl radical, a linear, branched or cyclic C1-20-alkyl radical or a C3-15-heterocycloalkyl radical and
the radicals R1 to R3 comprise, independently of one another, from 0 to 5 substituents which are inert under the reaction conditions and are selected from the group consisting of C1-20-alkoxy, hydroxy, halogen, C6-20-aryloxy, amino, C1-20-alkylamino and C2-20-dialkylamino.

14. The process of claim 12, wherein
R1 is a C11-20-arylalkyl radical having at least one fused ring system, a C10-20-aryl radical having at least one fused ring system or a C8-15-heteroaryl radical having at least one fused ring system,
R2 is a linear, branched or cyclic C1-20-alkyl radical or a C3-15-heterocycloalkyl radical and
R3 is hydrogen, a C7-20-arylalkyl radical, a C6-20-aryl radical, a C3-15-heteroaryl radical, a linear, branched or cyclic C1-20-alkyl radical or a C3-15-heterocycloalkyl radical and
the radicals R1 to R3 comprise, independently of one another, from 0 to 5 substituents which are inert under the reaction conditions and are selected from the group consisting of C1-20-alkoxy, hydroxy, halogen, C6-20-aryloxy, amino, C1-20-alkylamino and C2-20-dialkylamino.

15. The process of claim 12, wherein the amine of formula I is selected from the group consisting of 1-(1-naphthyl)ethylamine, 1-(2-naphthyl)ethylamine, 1-(1-naphthyl)propylamine, 1-(2-naphthyl)propylamine, 1-(1-naphthyl)isobutylamine, 1-(2-naphthyl)isobutylamine, 1-(1-naphthyl)-n-butylamine, 1-(2-naphthyl)-n-butylamine, 1-phenylethylamine, 1-phenylpropylamine, 1-phenylisobutylamine, 1-phenyl-n-butylamine, 1-(3-methoxyphenyl)ethylamine, 1-(2-methoxyphenyl)ethylamine, 1-(4-methoxyphenyl)ethylamine, 1-(3-methylphenyl)ethylamine, 1-(2-methylphenyl)-ethylamine, 1-(4-methylphenyl)ethylamine, 1-(3-chlorophenyl)ethylamine, 1-(2-chlorophenyl)ethylamine, 1-(4-chlorophenyl)ethylamine, 1-(3-fluorophenyl)ethylamine, 1-(2-fluorophenyl)ethylamine, 1-(4-fluorophenyl)ethylamine, 1-(3-hydroxyphenyl)ethylamine, 1-(2-hydroxyphenyl)ethylamine, 1-(4-hydroxyphenyl)-ethylamine, 1-(3-t-butylphenyl)ethylamine, 1-(2-t-butylphenyl)ethylamine, 1-(4-t-butylphenyl)ethylamine, 2-amino-1-phenylpropane, 2-amino-1-(p-hydroxyphenyl)-propane, 2-amino-1-(p-trifluoromethylphenyl)propane, 1-aminoindane, 1-aminotetralin and 2-aminotetralin.

16. The process of claim 12, wherein the amine of formula I is selected from the group consisting of 1-(1-naphthyl)ethylamine, 1-(2-naphthyl)ethylamine, 1-(1-naphthyl)propylamine, 1-(2-naphthyl)propylamine, 1-(1-naphthyl)isobutylamine, 1-(2-naphthyl)isobutylamine, 1-(1-naphthyl)-n-butylamine and 1-(2-naphthyl)-n-butylamine.

17. The process of claim 2, wherein the hydrogenation/dehydrogenation catalyst comprises an inert support material.

18. The process of claim 2, wherein the hydrogenation/dehydrogenation catalyst additionally comprises at least one further active component selected from the group consisting of zirconium, molybdenum and palladium, which further active component is present entirely or partly in a form of an oxide, before being activated by a reducing agent.

19. The process of claim 3, wherein the hydrogenation/dehydrogenation catalyst additionally comprises at least one further active component selected from the group consisting of zirconium, molybdenum and palladium, which further active component is present entirely or partly in a form of an oxide, before being activated by a reducing agent.

20. The process of claim 17, wherein the hydrogenation/dehydrogenation catalyst additionally comprises at least one further active component selected from the group consisting of zirconium, molybdenum and palladium, which further active component is present entirely or partly in a form of an oxide, before being activated by a reducing agent.
